(19) United States
(12) Patent Application Publication
(10) Pub. No.: US 2023/0320810 A1
(43) Pub. Date: Oct. 12, 2023

(54) LIVING BODY COMPRESSING CLIP

(71) Applicants: NATIONAL UNIVERSITY CORPORATION KOCHI UNIVERSITY, Kochi-shi, Kochi (JP); NIREC CORPORATION, Kochi-shi, Kochi (JP)

(72) Inventors: Takayuki SATO, Kochi-shi (JP); Tetsuo SUMIDA, Kochi-shi (JP)

(73) Assignees: NATIONAL UNIVERSITY CORPORATION KOCHI UNIVERSITY, Kochi-shi, Kochi (JP); NIREC CORPORATION, Kochi-shi, Kochi (JP)

(21) Appl. No.: 17/612,036

(22) PCT Filed: Nov. 30, 2020
(86) PCT No.: PCT/JP2020/044546

§ 371 Date: Jun. 27, 2023

(51) Int. Cl. A61B 90/00; A61B 17/122; A61B 17/128

(52) U.S. Cl. CPC A61B 90/39; A61B 17/1227; A61B 17/1285; A61B 2017/12004

(57) ABSTRACT

A living body compressing clip 100 includes a clip body 1 with metallic arm parts 5a and 5b configured to hold a living body tissue. The clip body 1 includes compressing pieces 10 that protrude from distal end parts of the arm parts 5a and 5b in a long-side direction of the arm parts 5a and 5b. The compressing pieces 10 are formed from a flexible resin and hold a fluorescent dye. According to the living body compressing clip 100, the compressing pieces 10 causes a vascular network to collapse while holding the living body tissue by the arm parts 5a and 5b. Therefore, when the arm parts 5a and 5b hold the mucosal tissue of the tubular organ and excitation light is applied thereto, the fluorescence emitted by the compressing pieces 10 can be satisfactorily confirmed from the serosal side.

### TECHNICAL FIELD

The present invention relates to a clip useful as a marker for specifying a position of an affected part.

### BACKGROUND ART

Conventionally, a metallic clip which is mounted on an endoscope clip device for use is widely used as a hemostatic clip or a marker clip for specifying a position of an affected part during a surgical operation. Examples of such a clip may include a clip which is molded by bending a flat spring made of metal such as stainless steel into a dogleg shape (Japanese character “ [symbol] ” shape), and a clip constituted by a metallic clip body molded in a dogleg shape (Japanese character “ [symbol] ” shape) and a fastener member which is externally fitted onto the clip body for fastening (Patent Literature 1).

After the mucosal tissue of the tubular organ is held by such a clip, a position of the clip needs to be confirmed from the serosal side. For this purpose, the present inventor has proposed that the fastener member of the clip is formed from a resin containing a fluorescent dye (Patent Literature 2) and that the clip body itself is formed from a resin containing a fluorescent dye (Patent Literature 3). When the mucosal tissue is held by these clips, the mucosal tissue is compressed, so that the blood is removed from a vascular network in the submucosal layer. Thus, when excitation light is applied to the clip from the serosal side to cause emission of the fluorescent dye, the emitted fluorescence is barely absorbed by the blood in the vascular network, making it possible to clearly confirm the position of the clip by observing the fluorescence from the serosal side.

### CITATION LIST

### Patent Literature

Patent Literature 1: Japanese Patent No. 6572229
Patent Literature 2: Japanese Patent No. 6161096
Patent Literature 3: Japanese Patent No. 6675674

### SUMMARY OF INVENTION

### Technical Problem

However, in the clip described in Patent Literature 2, the fastener member formed from the resin containing the fluorescent dye is required to have a specific shape for allowing the fastener member to compress the mucosal tissue. Further, in the clip described in Patent Literature 3, the clip body formed from the resin containing the fluorescent dye is required to have a specific shape capable of performing an opening and closing function of the clip. Thus, production methods of these clips must be significantly altered from the production method of conventional metallic clips.

The clip described in Patent Literature 3 comes with a problem of having a weak force for holding the living body tissue as compared with the conventional metallic clip.

Regarding this, an object of the present invention is to produce a clip, which is mounted on an endoscope clip device for use and can be positively confirmed by emission of fluorescence after being placed inside the body, in a more simplified manner by utilizing a configuration of a conventional metallic clip.

### Solution to Problem

The present inventor has conceived that, when compressing pieces, formed from a flexible resin containing a fluorescent dye, are attached to distal end parts of arm parts of a metallic clip body for holding a living body tissue so as to protrude in a long-side direction of the arms, the living body tissue can be compressed by protrusion parts of the compressing pieces while being held by the clip and that the clip body to which the compressing pieces are attached can be configured in the same manner as a conventionally used metallic clip body. As a result, the present inventor has conceived the present invention.

That is, the present invention provides a living body compressing clip which includes a clip body with metallic arm parts configured to hold a living body tissue, in which the clip body includes compressing pieces that protrude from distal end parts of the arm parts in a long-side direction of the arm parts, the compressing pieces are formed from a flexible resin and hold a fluorescent dye, and the compressing pieces compress the living body tissue while the arm parts hold the living body tissue.

### Advantageous Effects of Invention

The clip of the present invention can be easily produced simply by attaching the compressing pieces formed from the flexible resin and which hold the fluorescent dye to the distal end parts of the arm parts of the metallic clip body conventionally used as a hemostatic clip or a marker clip used during a surgical operation.

Further regarding this clip, the compressing pieces include parts protruded from the distal end parts of the arm parts and are flexible. Accordingly, when the living body tissue is held by the arm parts, the protrusion parts of the compressing pieces become bent and the living body tissue is compressed in the thickness direction thereof by the bent parts. In particular, in a case where the compressing pieces have elasticity, the living body tissue is compressed by the compressing pieces by virtue of an elastic force trying to recover from the bending. Thus, for example, when a mucosal tissue of a tubular organ is held by the arm parts, the protrusion parts of the compressing pieces are bent and compress the mucosa, causing a reduction in the thickness of the mucosa. As a result, when excitation light is applied to the compressing pieces from the serosal side to cause emission of fluorescence of the fluorescent dye held by the compressing pieces, the emission of the fluorescence can be satisfactorily confirmed from the serosal side.

That is, when the mucosal tissue is compressed, a vessel in a vascular network of arteries or veins in the submucosal layer collapses, resulting in removal of the blood as well as hemoglobin from the vessel. Thus, in the case where the fluorescent dye that emits red-to-near-infrared light upon irradiation with excitation light is used, the excitation light is absorbed by the fluorescent dye of the compressing pieces with hardly any absorption by hemoglobin, and the fluorescence emitted from the fluorescent dye is emitted to the serosal side with hardly any absorption by hemoglobin. This makes it possible to satisfactorily visually recognize the emission of the compressing pieces of the living body compressing clip attached to the mucosa of the tubular organ from the outside of the tubular organ.

### BRIEF DESCRIPTION OF DRAWINGS

FIG. 1 is a perspective view of a clip body, in an open state, used in a living body compressing clip in an example.

FIG. 2 is a side view of the clip body, in an open state, used in the living body compressing clip in the example.

FIG. 3A is a top view of the clip body used in the living body compressing clip in the example in a state of being closed by a fastener member.

FIG. 3B illustrates a side view as seen from a direction A and a side view as seen from a direction B of the clip body used in the living body compressing clip in the example in a state of being closed by the fastener member.

FIG. 4 is a side view of a living body compressing clip, in an open state, in the example.

FIG. 5A is a top view of the living body compressing clip, in a closed state, in the example.

FIG. 5B illustrates a side view as seen from a direction A and a side view as seen from a direction B of the living body compressing clip, in a closed state, in the example.

FIG. 6 is a diagram explaining flexibility of compressing pieces of the living body compressing clip in the example.

FIG. 7A is a diagram explaining a use method of the living body compressing clip in the example using an endoscope device.

FIG. 7B is a diagram explaining the use method of the living body compressing clip in the example using the endoscope device.

FIG. 7C is a diagram explaining the use method of the living body compressing clip in the example using the endoscope device.

FIG. 7D is a diagram explaining the use method of the living body compressing clip in the example using the endoscope device.

FIG. 8A is a diagram explaining how the living body compressing clip in the example acts on the mucosa.

FIG. 8B is a diagram explaining how the living body compressing clip in the example acts on the mucosa.

FIG. 8C is a diagram explaining how the living body compressing clip in the example acts on the mucosa.

FIG. 8D is a diagram explaining how the living body compressing clip in the example acts on the mucosa.

### DESCRIPTION OF EMBODIMENTS

The present invention will be described in detail below with reference to drawings. Note that, in each of the drawings, like reference symbols indicate like or equivalent components.

### <Overall Configuration of Living Body Compressing Clip>

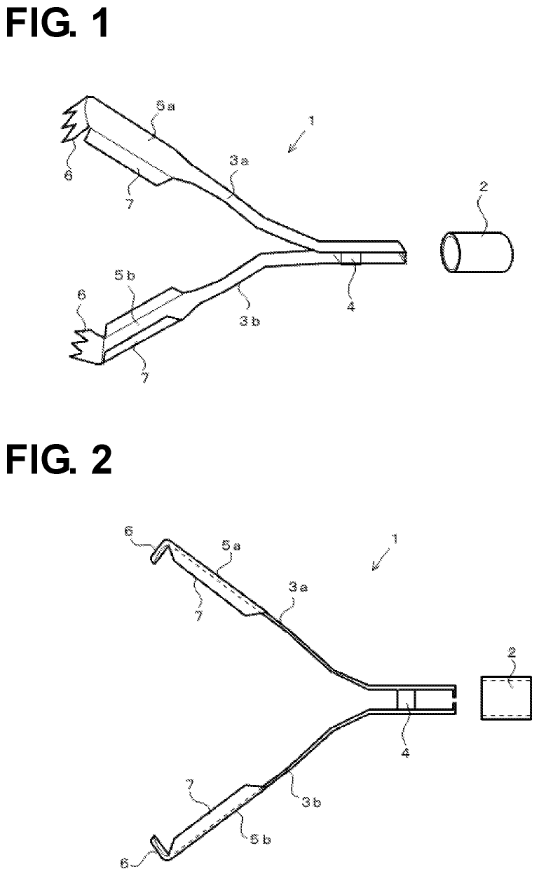

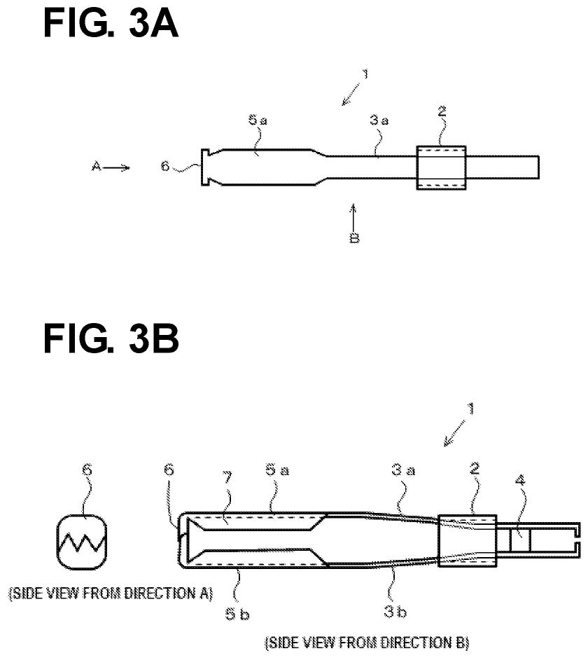

FIG. 1 is a perspective view of a clip body 1 and a fastener member 2 used in a living body compressing clip in an example of the present invention. FIG. 2 is a side view thereof. FIG. 3A is a top view of the clip body 1 in a state of being closed by the fastener member 2 fitted to the clip body 1, and FIG. 3B illustrates a side view as seen from a direction A and a side view as seen from a direction B thereof.

### <Clip Body>

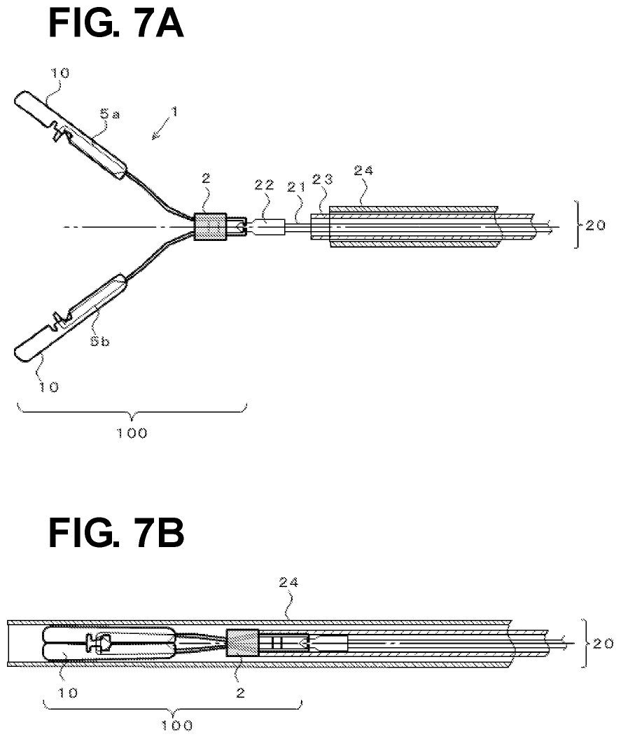

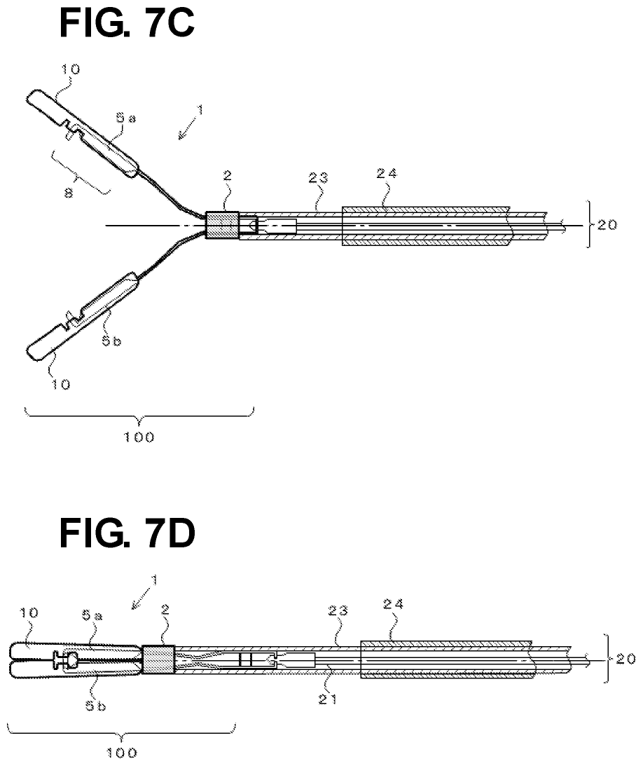

The living body compressing clip in the present example is mounted on an endoscope clip device (applier) and used as a clip for a living body tissue. The clip body 1 is formed by a pair of flat springs 3a and 3b obtained by bending strip-shaped metal plates. One end sections of these flat springs 3a and 3b are formed with a narrow width and the other end sections are formed with a wide width. The narrow end sections are bent in an L-shape, and notches (not shown) to be engaged with a connecting part 22 (FIG. 7A) of a manipulation wire 21 of the applier are formed on these bent sections. Further, a connecting member 4 is provided near the bent sections as needed to facilitate an engaging operation of the clip body 1 and the connecting part 22 of the manipulation wire 21 by keeping a constant distance between the pair of flat springs 3a and 3b. The connecting member 4 can be formed using the same material as that for the pair of flat springs 3a and 3b. The connecting member 4 can be attached by a method of screwing, fastening, welding, bonding, or the like.

The present invention is not limited to the configuration where the clip body 1 is formed by the pair of flat springs 3a and 3b. For example, as described in FIG. 3 in Japanese Patent No. 6572229 (Patent Literature 1), flat springs molded in a V-shape by bending one piece of strip-shaped metal plate at its central part may be used as the clip body 1. As described in FIG. 1 in WO2017/068970, a flat spring bent in a U-shape may be used as the clip body 1. Alternatively, for example, as described in Japanese Patent No. 4145149, the clip body 1 may be configured in such a manner that a pair of the arm parts 5a and 5b are intersected with each other.

The wide width sides of the pair of flat springs 3a and 3b constitute arm parts 5a and 5b where a distance between end sections of the pair of flat springs 3a and 3b is increased. The distal end parts of the arm parts 5a and 5b are bent in an L-shape to form claw parts 6. The claw parts 6 directly bite into a living body tissue to hold it. In the present example, the claw parts 6 have a triangular wave shape and are formed in such a manner that the triangular wave shapes of the claw parts 6 of the pair of flat springs 3a and 3b are engaged with each other (FIG. 3B) to improve the bite of the claw parts 6 into the living body tissue.

Note that, in the present invention, the shape of the claw parts 6 is not limited to the triangular wave shape. For example, the claw parts 6 may have a rectangular wave shape and the claw parts may be formed in a trapezoidal shape, a rectangular shape, or the like. In the present example, the claw parts of the pair of arm parts 5a and 5b are formed so as to abut against each other when the clip body 1 is closed. However, for example, as described in Japanese Patent No. 6233146, the claw parts of the pair of arm parts 5a and 5b may be configured to overlap with each other when the clip body 1 is closed.

Side parts 7 of the pair of arm parts 5a and 5b stand up toward the other corresponding arm parts 5b and 5a to constitute L-shaped bent parts. The side parts 7 of the arm parts 5a and 5b are bent in this manner as needed in order to increase rigidity of the arm parts 5a and 5b.

Note that the present invention is not limited to the configuration where the clip body 1 includes the pair of arm parts 5a and 5b. The clip body 1 may include three or more arm parts.

### <Fastener Member>

The fastener member 2, also referred to as a fastening ring, opens the clip body 1 by being positioned on the side of the connecting member 4 of the clip body 1 (FIG. 2) and closes the clip body 1 by being positioned on the side of the claw parts 6 (FIG. 3B). The fastener member 2 in the present example is formed by a metallic cylindrical member. As the fastener member 2, a member obtained by winding a wire in a coil shape, a member having a cross section formed in a C-shape perpendicular to the long-side direction, or the like may be used.

### <Compressing Piece>

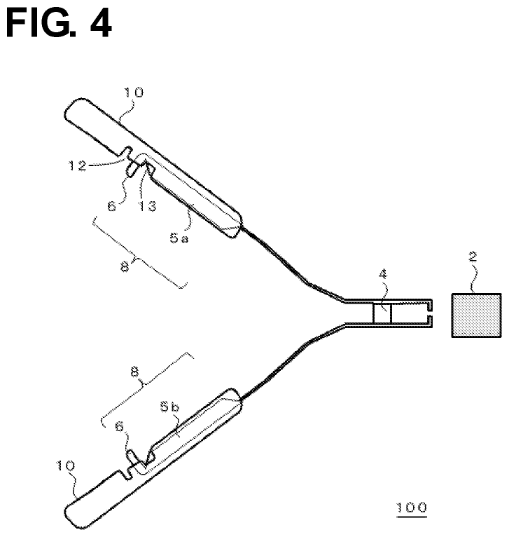

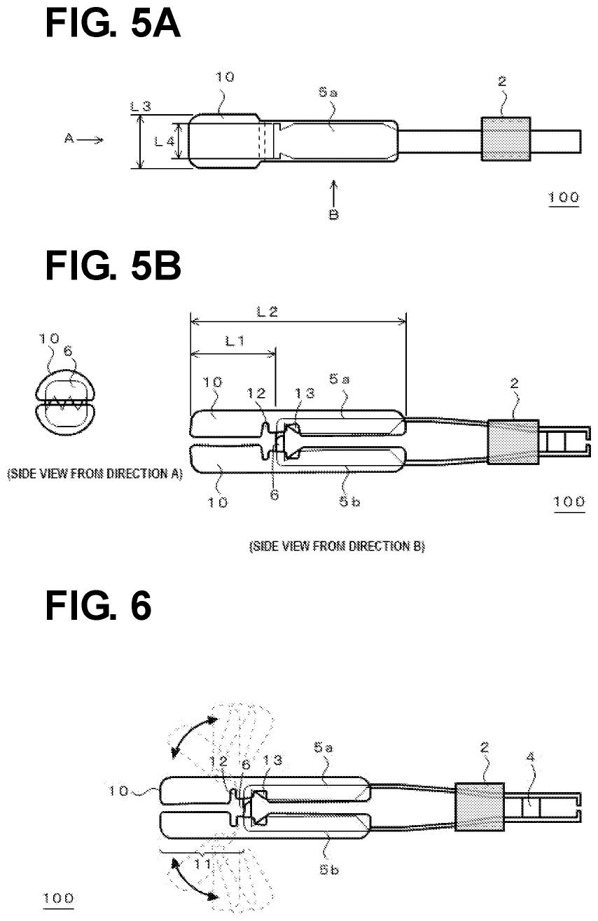

FIG. 4 is a side view of a living body compressing clip 100 of an example of the present invention in which the clip body 1 is opened. This living body compressing clip 100 includes compressing pieces 10 formed from a flexible resin at the distal end parts of the arm parts 5a and 5b of the clip body 1 described above. FIG. 5A is a top view of this living body compressing clip 100 in which the clip body 1 is closed by being fastened by the fastener member 2. FIG. 5B illustrates a side view as seen from a direction A and a side view as seen from a direction B. Note that the fastener member 2 is painted with gray color to facilitate the understanding of the drawing.

The compressing pieces 10, as a characteristic configuration of the present invention, protrude from the distal end parts (the claw parts 6) of the arm parts 5a and 5b in the long-side direction of the arm parts 5a and 5b. These compressing pieces 10 are disposed on the clip body 1 so as not to prevent the holding of the living body tissue by the arm parts 5a and 5b. That is, the arm parts 5a and 5b have holding parts 8 (in FIG. 4) that apply a holding pressure to the living body tissue when the living body tissue is held by the arm parts 5a and 5b, and the compressing pieces 10 are disposed along the back side of the arm parts 5a and 5b (in other words, on the opposite side of the holding parts 8) so that the holding parts 8 are exposed without being covered by the compressing pieces 10.

The compressing pieces 10 are formed from a flexible resin and hold a fluorescent dye. Such compressing pieces 10 can be formed from, for example, a flexible resin containing a fluorescent dye. Alternatively, a coating film containing a fluorescent dye may be formed on the surface of the compressing pieces formed from a flexible resin.

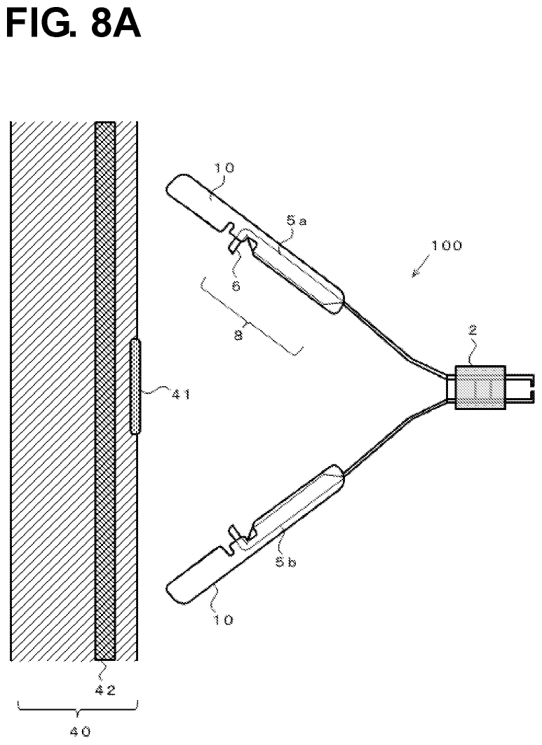

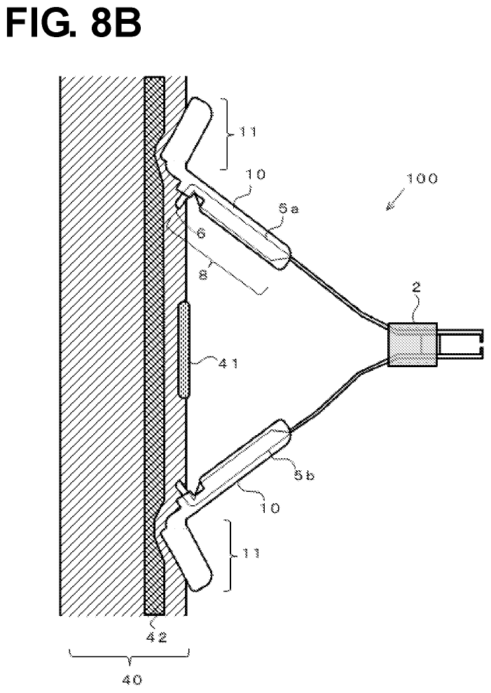

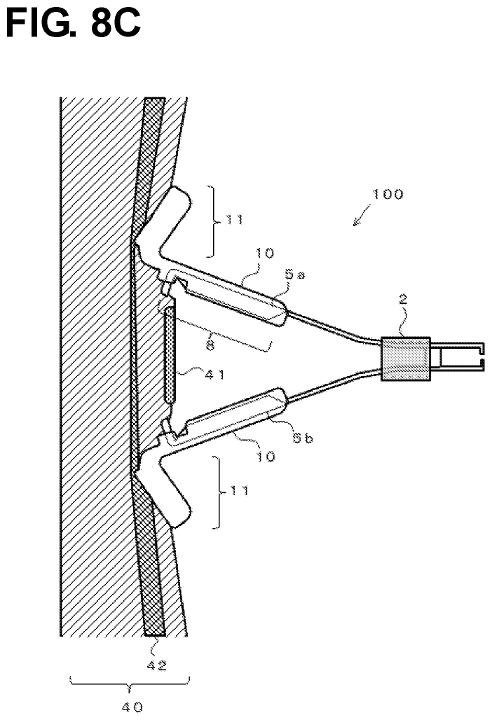

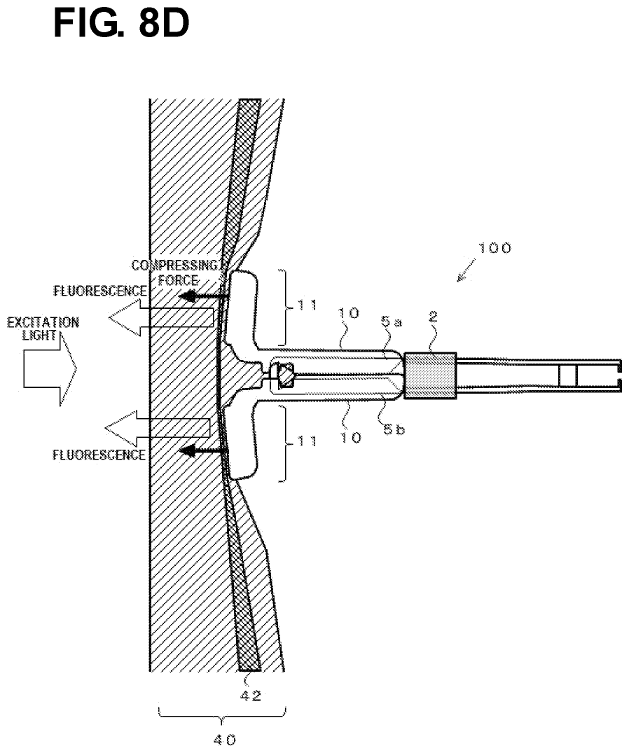

The compressing pieces 10 formed from the flexible resin allow protrusion parts 11 of the compressing pieces 10 protruding from the distal end parts 6 of the arm parts 5a and 5b to be bent as indicated by broken lines in FIG. 6. Thus, as described below, the protrusion parts 11 of the compressing pieces 10 are bent when the arm parts 5a and 5b of the clip body 1 hold the living body tissue. Then, the bent parts compress the living body tissue in the thickness direction thereof (the long-side direction of the arm parts 5a and 5b holding the living body tissue) (FIG. 8D). The compressing pieces 10 preferably have elasticity, so that the living body tissue is strongly compressed in the thickness direction thereof by an elastic force trying to recover from the bending of the compressing pieces 10. As a result, the blood in the vascular network in the submucosal layer is removed, and, when excitation light is applied to the compressing pieces 10 from the serosal side to cause emission of fluorescence from the fluorescent dye held by the compressing pieces 10, this fluorescence can be satisfactorily confirmed from the serosal side.

### (Shape of Compressing Pieces)

The compressing pieces 10 in the present example have a generally strip shape and include narrow parts 12 near the distal end parts 6 of the arm parts 5a and 5b outside the distal end parts 6 (a side far from the connecting member 4). The compressing pieces 10 also include narrow parts 13 inside the distal end parts 6 (a side close to the connecting member 4). In the present invention, the narrow parts of the compressing pieces 10 are appropriately provided as necessary near the distal end parts of the arm parts in order to adjust the compressing force to the living body tissue applied by the bent compressing pieces 10. Thus, only either of the outside narrow parts 12 or the inside narrow parts 13 may be provided or both may be omitted.

A protrusion length L1 (FIG. 5B) of the compressing pieces 10 from the distal end parts of the arm parts 5a and 5b is preferably set to from 2 to 10 mm, as setting it too short makes it difficult to obtain a sufficient compressing force by the compressing pieces 10, while setting it too long makes it difficult to hold the living body tissue by the clip body. A total length L2 of the compressing pieces 10 is preferably set to from 5 to 13 mm, as setting it too short leads to insufficient mounting strength of the compressing pieces 10 to the arm parts 5a and 5b, while setting it too long makes it difficult to use the living body compressing clip 100 in endoscope clip devices.

A width L3 (FIG. 5A) in a top view of the compressing pieces 10 is preferably set to be longer than a width L4 of the distal end parts of the clip body by 0.4 to 1.0 mm or set between 1.4 to 2.6 mm, as setting it too short makes it difficult to obtain a sufficient compressing force by the compressing pieces 10, while setting it too long makes it difficult to mount the living body compressing clip 100 on endoscope clip devices.

### (Flexible Resin for Forming Compressing Pieces)

The flexible resin for forming the compressing pieces 10 preferably has acid resistance sufficient to resist gastric acid so that the flexible resin is not denatured even if it is placed in the digestive tract for a long period of time. Examples of such a flexible resin may include resins used for medical devices such as a soft polyvinyl chloride, a thermoplastic polyurethane, silicone, and an ethylene-vinyl acetate copolymer.

The flexible resin for forming the compressing pieces 10 preferably has adequate hardness and elasticity so that the bent protrusion parts 11 of the compressing pieces 10 can effectively compress the living body tissue when the arm parts 5a and 5b of the clip body 1 hold the living body tissue.

The hardness of the compressing pieces 10 is preferably within a range of A10 to A90 in Shore A hardness (JIS K 6253) or within a range of D40 to D70 in Shore D hardness (JIS K 6253) measured by a durometer. The hardness of the compressing pieces 10 is preferably appropriately selected in accordance with the shape, size, or the like of the compressing pieces 10.

For the elasticity of the compressing pieces 10, the bending elastic modulus (JIS K 7203) is preferably from 4 to 200 MPa.

### (Fluorescent Dye)

The fluorescent dye to be held by the compressing pieces emits fluorescence within a red-to-near-infrared wavelength region of 600 to 1400 nm, and preferably fluorescence within a red or near-infrared wavelength region of 700 to 1100 nm. Light in such a wavelength region has a high transmittance to human tissues such as skin, fat, and muscle, and, for example, it can satisfactorily reach from the mucosa to the serosal surface of the human tubular tissue such as the rectum.

Examples of the fluorescent dye emitting the fluorescence in the above-mentioned wavelength region may include riboflavin, thiamine, nicotinamide adenine dinucleotide (NADH), indocyanine green (ICG), azo-boron complex compounds described in Japanese Patent Application Laid-Open No. 2011-162445, dyes with a condensed ring structure described in WO2016/132596, dyes with a boron dipyrromethene structure described in Japanese Patent No. 5177427, dyes chemically bonded to silica particles described in Japanese Patent Application Laid-Open No. 2020-74905, and phthalocyanine-based dyes described in Japanese Patent Application Laid-Open No. 2020-105170.

As a concrete mode of allowing the fluorescent dye to be held by the compressing pieces 10, for example, the fluorescent dye can be included in the flexible resin forming the compressing pieces 10 or a coating film containing the fluorescent dye can be formed on the surface of the compressing pieces formed from the flexible resin. In the case where the fluorescent dye is included in the flexible resin forming the compressing pieces 10, the preferable concentration of the fluorescent dye can be set in accordance with the type or the like of the fluorescent dye and the resin, and, normally, it is preferably set to from 0.001 to 1 mass %.

As a method for allowing the flexible resin to include the fluorescent dye, for example, the fluorescent dye is kneaded with the flexible resin using a biaxial kneader.

A contrast agent such as barium sulfate may be added to the flexible resin as needed. In this case, even if the living body compressing clip holding the mucosa in the living body comes off from the mucosa, the living body compressing clip in the living body can be tacked by using X-ray photography.

### (Method for Forming Compressing Pieces)

The compressing pieces can be obtained by, for example, molding the flexible resin kneaded with the fluorescent dye into a prescribed shape according to extrusion molding or injection molding, and then subjecting the molded product to processing such as standard-length cutting, corner removal, and concave formation. The compressing pieces thus obtained are bonded to the clip body 1 using an adhesive. Alternatively, insertion molding may be performed using the clip body to obtain the compressing pieces 10 fixed to the clip body.

### <Use Method of Living Body Compressing Clip>

As a use method of the living body compressing clip 100, first, as shown in FIG. 7A, the living body compressing clip 100 including the clip body 1 having the compressing pieces 10 and the fastener member 2 is attached to a clip sheath 20 of an endoscope applier. As the clip sheath 20, for example, a clip sheath including an inner sheath 23, an outer sheath 24, and the manipulation wire 21 described in Japanese Patent No. 4388324, Japanese Patent No. 5045484, or the like can be used. A commercially available product thereof can be used. As a method for attaching the living body compressing clip 100 to the clip sheath 20, for example, as shown in FIG. 7A, the connecting part 22 of the manipulation wire 21 is hooked on the end part of the clip body 1 on the side of the connecting member 4. In the case where the clip body 1 is formed by a flat spring bent in a V-shape, a U-shape, or the like, a hook may be used as described in Japanese Patent No. 5781347.

When the living body compressing clip 100 is pulled into the outer sheath 24 by operation of the applier, as shown in FIG. 7B, the clip body 1 is closed. When the clip body 1 is pushed out from the outer sheath 24, the clip body 1 on the side of the holding parts 8 is opened. As shown in FIG. 7C, when the inner sheath 23 is brought into contact with the fastener member 2 and the fastener member 2 is slid to the side of the holding parts 8, the clip body 1 is gradually closed, and, eventually, as shown in FIG. 7D, the open clip body 1 is completely closed.

Next, a description is given of a case where an affected part of the mucosa of the tubular organ is held by the living body compressing clip 100. The clip body 1 is attached to the manipulation wire 21 and the living body compressing clip 100 is pulled into the outer sheath 24. The clip sheath 20 is inserted into the tubular organ in a state in which the clip body 1 is closed. The living body compressing clip 100 is pushed out from the outer sheath 24 by operating the applier to open the arm parts 5a and 5b of the clip body 1 at the side of the holding parts 8 near an affected part 41 (FIG. 8A). Next, the living body compressing clip 100 is brought into contact with a mucosa 40 and the fastener member 2 is moved to the side of the holding parts 8. The clip body 1 is closed in accordance with the amount of movement and the protrusion parts 11 of the compressing pieces 10 become bent (FIG. 8B and FIG. 8C). When the clip body 1 is completely closed, the mucosa 40 near the affected part 41 is held by the holding parts (FIG. 8D). At this time, the protrusion parts 11 of the compressing pieces 10 are bent as shown in FIG. 8D and the compressing pieces 10 compress the mucosa 40 in a direction where the thickness of the mucosa 40 is reduced. In particular, in the case where the compressing pieces 10 have elasticity, the elastic force directed in a direction of recovering from the bending of the compressing pieces 10 acts in the direction where the thickness of the mucosa 40 is reduced. Thus, a vascular network 42 in the submucosal layer collapses and the blood as well as hemoglobin are removed from the vessel.

Thus, when excitation light in a red-to-near-infrared wavelength region is applied to the outside (serosal side) of the tubular organ, the applied excitation light is absorbed by the fluorescent dye contained in the flexible resin forming the compressing pieces 10 with hardly any absorption or inhibition by hemoglobin, causing the fluorescent dye to emit fluorescence in a red-to-near-infrared wavelength region. This fluorescence is emitted to the outside of the tubular organ with hardly any absorption or inhibition by hemoglobin. Thus, this fluorescence can be satisfactorily observed from the outside of the tubular organ, making it possible to determine the position of the living body compressing clip holding inside the tubular organ and thereby to specify the position of the affected part 41.

In this case, as a method for applying the excitation light to the serosal side of the tubular organ, the excitation light may be applied to the serosa of the tubular organ exposed by performing a thoracotomy or a laparotomy. Alternatively, a laparoscope (surgical endoscope) may be inserted from a hole made in the wall of the thorax or the abdomen and the excitation light in the red-to-near-infrared wavelength region may be applied to the serosal surface or the peritoneal surface of the tubular organ while the serosal surface or the peritoneal surface of the tubular organ is being observed.

Note that, in a case where the fluorescence observed from the outside of the tubular organ is not visible light, a light-emitting site can be easily specified by observing the fluorescence through a known infrared-visible light conversion glass or imaging the tubular organ from the outside and visualizing the fluorescence by image processing.

This living body compressing clip 100 can be attached to the mucosa of the digestive tract such as the esophagus, the stomach, or the large intestine, the tracheae mucosa, the bladder mucosa, the uterine mucosa, and the like, thereby making it possible to mark affected sites of these tubular organs with certainty.

### REFERENCE SIGNS LIST

1 clip body
2 fastener member (fastening ring)
3a, 3b flat spring
4 connecting member
5a, 5b arm part
6 claw part
7 side part
8 holding part
10 compressing piece
11 protrusion part
20 clip sheath
21 manipulation wire
22 connecting part
23 inner sheath
24 outer sheath
40 mucosa
41 affected part
42 vascular network
100 living body compressing clip

1. A living body compressing clip comprising a clip body with metallic arm parts configured to hold a living body tissue, wherein the clip body includes compressing pieces that protrude from distal end parts of the arm parts in a long-side direction of the arm parts, the compressing pieces are formed from a flexible resin and hold a fluorescent dye, and the compressing pieces compress the living body tissue while the arm parts hold the living body tissue.

2. The living body compressing clip according to claim 1, wherein holding parts configured to apply a holding pressure to the living body tissue when the living body tissue is held are exposed without being covered by the compressing pieces.

3. The living body compressing clip according to claim 1 or 2, wherein the compressing pieces have a Shore A hardness (JIS K 6253) of A10 to A90 or a Shore D hardness (JIS K 6253) of D40 to D70.

4. The living body compressing clip according to any one of claims 1 to 3, wherein the compressing pieces have elasticity.

5. The living body compressing clip according to claim 4, wherein the compressing pieces have a bending elastic modulus of 4 to 200 MPa.

6. The living body compressing clip according to any one of claims 1 to 5, wherein a protrusion length of the compressing pieces is 2 to 10 mm.

7. The living body compressing clip according to any one of claims 1 to 6, wherein the compressing pieces include narrow parts near the distal end parts of the arm parts.

8. The living body compressing clip according to any one of claims 1 to 7, wherein the fluorescent dye emits red-to-near-infrared light upon irradiation with excitation light.
